(19) United States
(12) Patent Application Publication
(10) Pub. No.: US 2004/0210241 A1
(43) Pub. Date: Oct. 21, 2004

(54) Method and apparatus for stitching

(76) Inventors: Adam James, South Glamorgan (GB); Andrew Thomson, Leicestershire (GB)

Correspondence Address: DEMONT & BREYER, LLC, SUITE 250, 100 COMMONS WAY, HOLMDEL, NJ, 07733, US

(21) Appl. No.: 10/482,551

(22) PCT Filed: Jul. 4, 2002
(86) PCT No.: PCT/GB02/03086

(30) Foreign Application Priority Data
Jul. 4, 2001 (GB) 0116247.8

(51) Int. Cl. A61B 17/10

(52) U.S. Cl. 606/139

(57) ABSTRACT

There is disclosed a method for stitching, comprising penetrating a material to be stitched, delivering through the penetration an anchor, securing a first end of a thread by the anchor in the penetration, penetrating the material in a position spaced from the first penetration, delivering through the second penetration a second anchor, attaching the thread through the second penetration by the second anchor to the material, repeating the procedure as often as required until a final penetration, at which a final anchor is delivered and a second end of the thread secured by the final anchor thereto.

This invention relates to stitching, particularly, but not exclusively, to surgical suturing.

Material, e.g. soft tissue layers, can be stitched, or sutured, using thread which is passed through the tissue using a needle, as in conventional hand sewing, and knotted. As an alternative, also in surgery, staples are used. Each has its place. Conventional thread suturing is usually no problem for a skilled surgeon when the suturing site is readily accessible. In endoscopic procedures, however, where access is restricted, conventional stitching, and particularly knotting, is difficult. Staples are relatively easy to deploy, but have their own drawbacks, principally that they can misfire and migrate, and, being easy to deploy, tend to be overused, which may necessitate further surgical recovery procedures.

The present invention provides methods and apparatus for stitching which are of use in surgery, but which may also be useful in other areas, for example in textile stitching.

The invention comprises, in one aspect, a method for stitching, comprising penetrating a material to be stitched, delivering through the penetration an anchor, securing a first end of a thread by the anchor in the penetration, penetrating the material in a position spaced from the first penetration, delivering through the second penetration a second anchor, attaching the thread through the second penetration by the second anchor to the material, repeating the procedure as often as required until a final penetration, at which a final anchor is delivered and a second end of the thread secured by the final anchor thereto.

The anchors may be threaded on the thread. If they are loosely threaded, free to move along the thread, the spacing between penetrations may be chosen at will.

Penetration may be effected by a piercing element, An anchor delivery element delivers, at each penetration, an anchor through the material. After each penetration, the piercing and delivery elements may be withdrawn, leaving a length of thread through the penetration, secured on the anchor. A predetermined tension may be applied to the thread at each penetration to effect a desired tightness.

An embedding action may then follow to embed the anchor in the material. The embedding action may comprise a rotation, which may be through 180°. The embedding action may comprise a pulling action.

The anchor may be barbed, the better to retain it in its embedded position.

After the last anchor has been delivered, the thread may be severed.

A stitching tool may hold a plurality of anchors, the anchor delivery element advancing anchors one by one to successive penetrations.

The method may be used in surgery, and may be used for fixing soft tissue to soft tissue, or soft tissue to bone, in which case at least one suture may be implantable in bone. Two anchors may be implanted into bone through soft tissue to be fixed thereto, the suture thread extending between them being tightened to hold the tissue against the bone. Anchors with attached threads are already known primarily for fixing tissue to bone, though with the possibility of fixing tissue to tissue, see, for example, U.S. Pat. No. 6,024,758 and EP 1 199 036. As in those publications, the bone may be pre-drilled for the anchor.

The invention also comprises an anchor and thread assembly for use in the methods herein proposed, comprising a thread and a plurality of eyed anchors threaded thereon. The thread may be a surgical suture thread, and the anchors bio-compatible.

The invention also comprises an anchor for an assembly as proposed, having a hook-like configuration, having a barb at at least one end and an eye for a thread. The anchor may be made of a bio-compatible material, which may be a bio-absorbable material, such as an absorbable polymer such as polylactic acid or a copolymer of PGA-co-TMC or of PGA-co-PCL.

The anchor may, however, be made of metal or a non-absorbable polymer such as acetal.

The anchor may comprise a releasable biological factor, such as a drug, an antibiotic or an inducer or trigger for a natural healing factor.

The invention also comprises a stitching implement comprising an anchor reservoir for a plurality of anchors, a thread reservoir for a thread and a piercing implement operative to penetrate material to be stitched and an anchor delivery implement to deliver through the penetration one of said anchors with attached thread, the stitching implement being adapted to withdraw the delivery element and piercing implement from the penetration, leaving the anchor, and to repeat the procedure at spaced apart locations along the material.

The anchor reservoir may comprise a slideway on which anchors can be stacked, rather like staples in a stapler. The thread reservoir may comprise a spool holder. The anchor and thread reservoirs may hold a plurality of anchors threaded on a thread held in the thread reservoir.

The stitching implement may have a thread cutting knife, which may be extendable from the region of the thread and anchor reservoir into an operative position.

The stitching implement may be fashioned as an endoscopic surgical device, and may comprise an anchor indexing mechanism and a thread tension defining system operable from a handpiece.

Methods of stitching, especially in surgery, an anchor, an anchor and thread assembly and a stitching implement will now be described with reference to the accompanying drawings, in which:

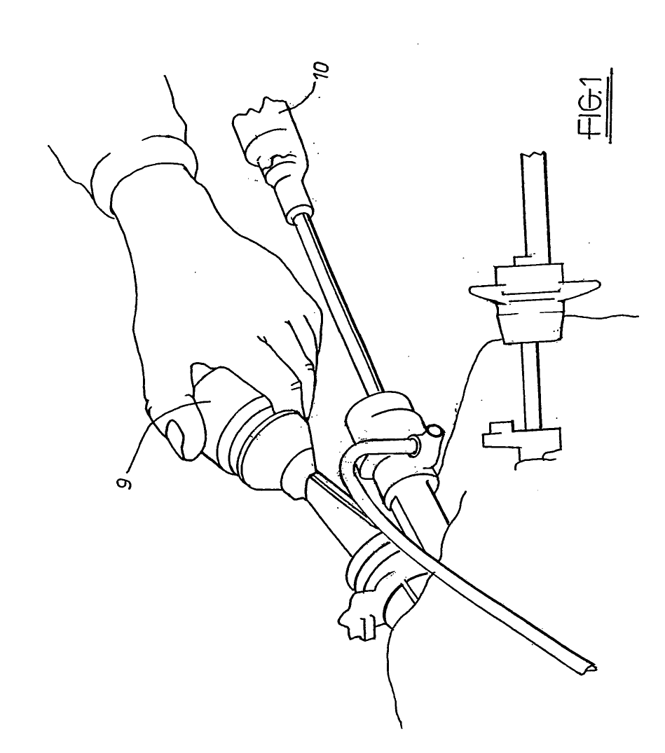

FIG. 1 is a view of a surgical site at which a stitching implement according to the invention is deployed;

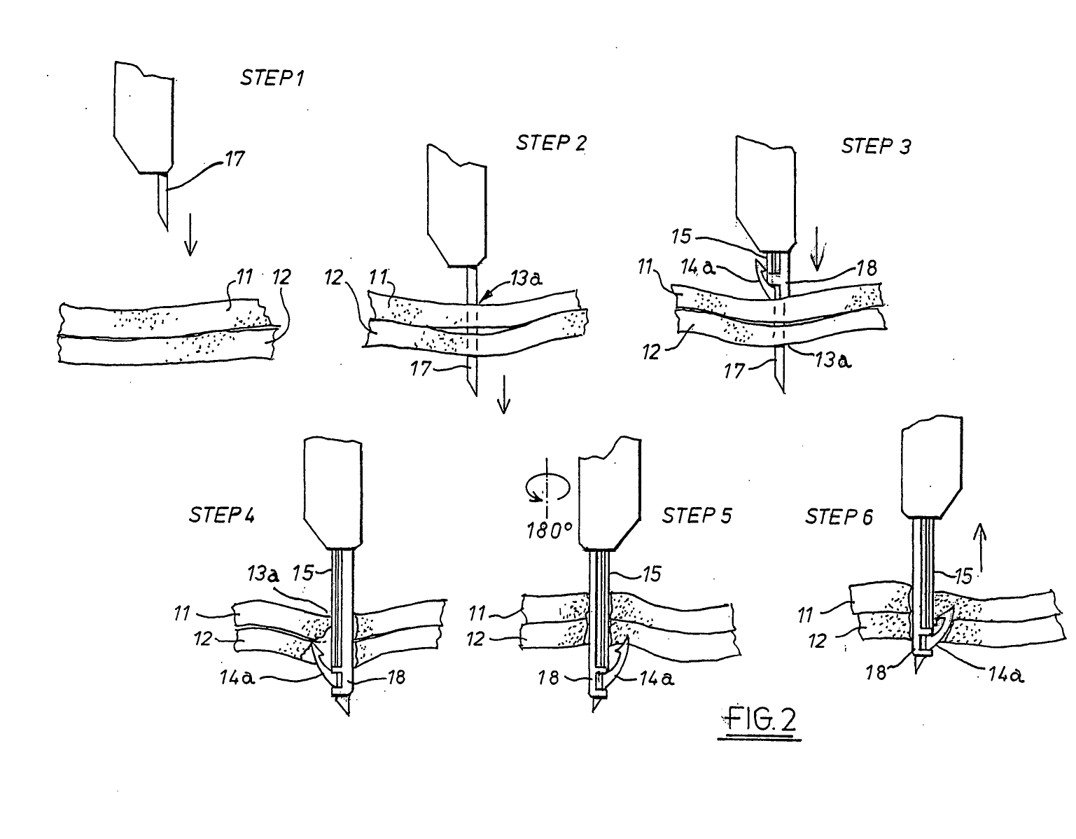

FIG. 2 is a diagrammatic depiction of steps 1 to 14 in a suturing procedure approximating soft tissues;

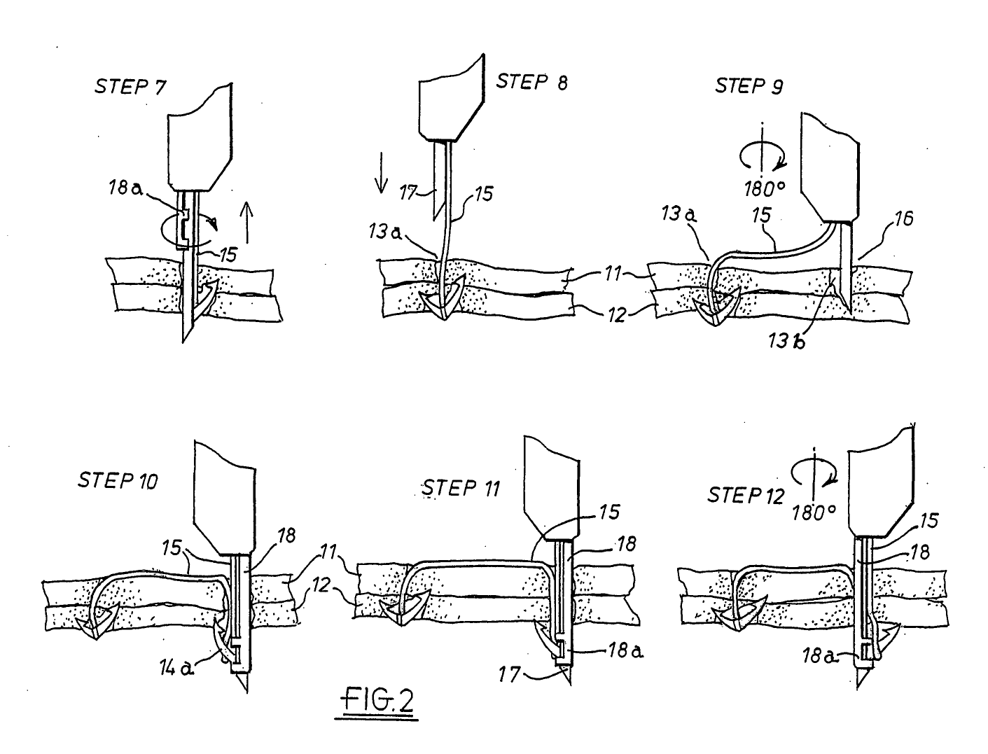

FIG. 3 is a cross section showing the suturing extending over three penetrations;

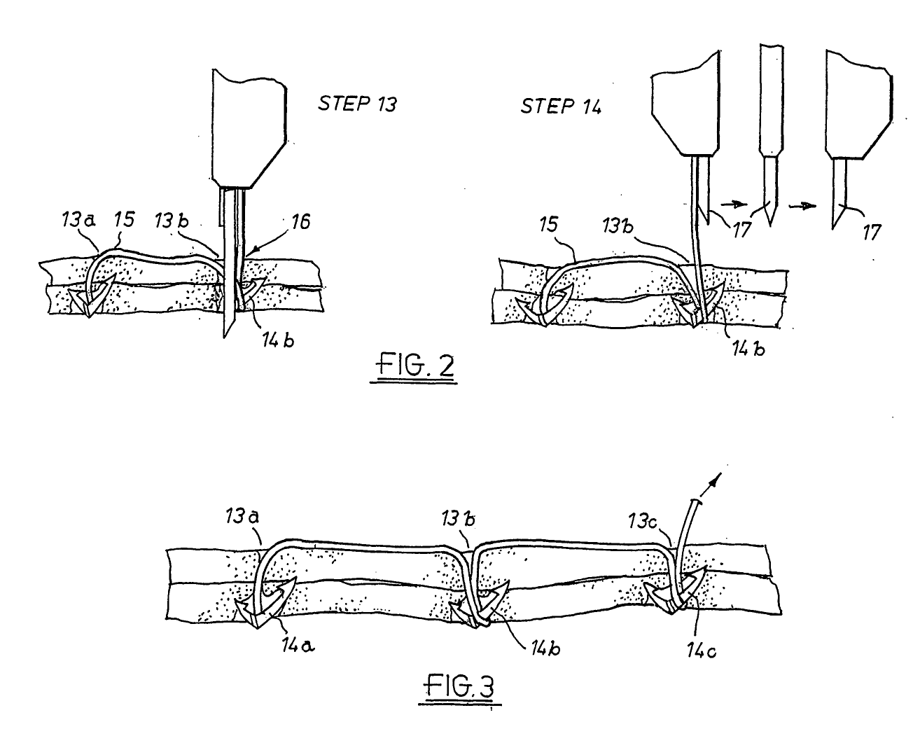

FIG. 4 is a lengthwise section through a stitching implement; and

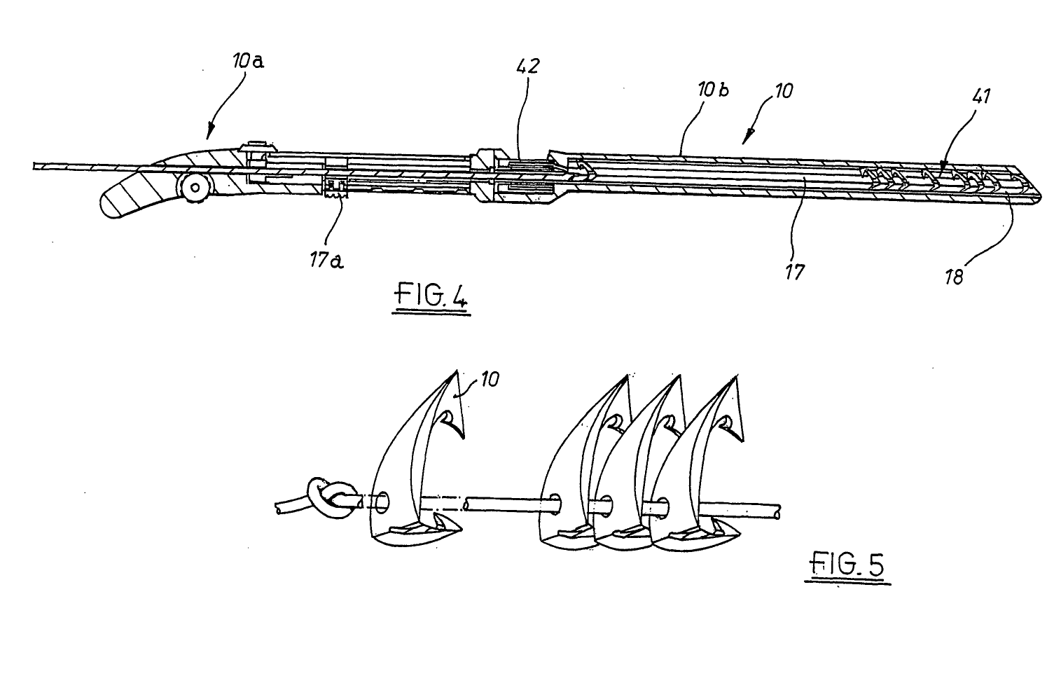

FIG. 5 is a view of anchors assembled on a thread for deployment by the implement of FIG. 4.

The drawings illustrate a method, using a stitching implement 10, for stitching comprising penetrating a material—in this case, soft tissues 11, 12 to be approximated—delivering through the penetration 13a an anchor 14a, securing a first end of a thread 15 by the anchor 14a in the penetration 13a, penetrating the material 11, 12 in a position 16 spaced from the first penetration 13a, delivering through the second penetration 13b, at position 16, a second anchor 14b, attaching the thread 15 through the second penetration 13b by the second anchor 14b to the material 11, 12, repeating the procedure as often as required until a final penetration 13c, at which a final anchor 14c is delivered and a second end of the thread secured by the final anchor 14c thereto.

FIG. 3 illustrates suturing extending over the three penetrations 13a, 13b, 13c.

As seen in FIG. 5, the anchors 14 are threaded on the thread 15, having eyes 14e for this purpose. The first anchor 14a is attached, as by a knot (as illustrated), or by a suitable adhesive, or by injection moulding or in any other appropriate fashion, to the first end of the thread 15. The other anchors are free to slide on the thread 15. The anchors 14 have barbs 21 at each end.

Penetration is effected by a piercing element 17. An anchor delivery element 18 delivers, at each penetration, an anchor 14 through the material. In the case of surgical suturing, of course, the piercing element 17 will actually bore through the tissues 11, 12. In other cases, the material may be pre-penetrated.

FIG. 2, Step 1, shows the piercing implement 17 projecting from the end of the stitching implement 10 to a preset depth set by a mechanism 118 (FIG. 4) in the handpiece 10a of the implement 10. Step 2 shows penetration by the piercing element 17. And Step 3 shows implantation of an anchor 14a, with thread 15 attached, by the delivery element 18, which is operated by an actuator 18a in the handpiece 10a. Step 4 shows the anchor 14a—which has a sharp leading edge 19 to extend the penetration—fully through the penetration. In Step 5, the whole implement 10 has been axially rotated through 180° by the surgeon, placing the barb 21 against a part of the tissue 12 that has not been damaged by the penetration and effectively wrapping the thread about the anchor so as to secure it frictionally, and in Step 6, the implement 10 has been pulled back to embed the anchor 14 in the tissue. Step 7 shows the anchor delivery element 18 withdrawn from the penetration, leaving the anchor 14a behind, embedded in the tissue. Next, at Step 8, the implement 10 is pulled back, allowing a length of thread 15 to be unwound from the spool (shown here diagrammatically—in fact, it will be in the handpiece).

The implement 10 is now rotated back through 180°, and moved along to the next penetration site—Step 9. In Step 10, another penetration has been effected, and, in Step 11, excess thread 15 is pulled back by the spool. In Step 12, the implement 10 is again rotated to place the barb against undamaged tissue. Step 13 is essentially a repetition of Steps 6 and 7. In Step 14, the implement is fully withdrawn again, ready for the next penetration. At the end of the procedure, the thread 15 is cut, and the implement fully withdrawn. An extendable knife, not shown, may be deployed from the implement to sever the thread 15, or a knife or other device my be deployed through another portal at the surgical site.

The facility to pull thread back means that even separated soft tissues can be drawn together, as when a section has been excised—the thread can simply be “reeled in” to approximate the tissues. For this, the implement need not be rotated, or rotated only slightly less than the 180°, at each penetration, to reduce the frictional resistance to the thread passing through the anchors

FIG. 1 illustrates a typical surgical site for endoscopic surgery having a number of portals through which devices can be inserted, including a laparoscope 9 and the stitching implement 10.

FIG. 4 shows one embodiment of stitching implement 10 in cross-section, comprising a handpiece 10a, an anchor reservoir 41 for a plurality of anchors 14, a thread reservoir comprising a spool holder 42, a piercing element 17 with a depth setting actuator 17a, and an anchor delivery element 18 which has a formation at its end 18a which releasably engages an anchor. The piercing implement and the delivery element can be withdrawn back into the implement 10, the delivery element engaging the next in line anchor 14. The implement 10 can be configured as a handpiece 10a with an attachable cassette 10b holding the supply of anchors and thread. The handpiece 10a will have various actuators and controls, such at the thread tension control and rods, perhaps with spring return features, to extend the piercing implement and the delivery element, as well as the suture cutting knife, if provided. The piercing implement may, of course, be adapted to cut the suture.

Lapariscopic surgical procedures in which the stitching method can be used effectively include Collis-Nissen Fundoplication, Palliative side-to-side Esophagogastrostomy, End-to-end Anastomosis, Fascia Closure and Ileal “J” Pouch procedures.

Tissue can be fixed to bone using anchors as taught in the US and EP references mentioned above, or any other suitable anchoring devices such as screws located in pre-drilled bores in the bone.

1-39. (canceled)

40. A method for stitching, comprising penetrating a material to be stitched, delivering through the penetration an anchor, securing a first end of a thread by the anchor in the penetration, penetrating the material in a position spaced from the first penetration, delivering through the second penetration a second anchor, attaching the thread through the second penetration by the second anchor to the material, repeating the procedure as often as required until a final penetration, at which a final anchor is delivered and a second end of the thread secured by the final anchor thereto.

41. A method according to claim 40, in which the anchors are threaded on the thread.

42. A method according to claim 40, in which the penetration is effected by a piercing element.

43. A method according to claim 40, in which an anchor is delivered through a penetration by an anchor delivery element.

44. A method according to claim 43, in which, after each penetration, the piercing and delivery elements are withdrawn, leaving a length of thread through the penetration secured to the anchor.

45. A method according to claim 40, in which the stitching implement effects an embedding action to embed the anchor in the material.

46. A method according to claim 45, in which the embedding action comprises a rotation of the implement.

47. A method according to claim 46, in which the rotation is through 180°.

48. A method according to claim 46, in which the embedding action comprises a pulling action.

49. A method according to claim 40, in which the anchor is barbed.

50. A method according to claim 40, in which, after the last anchor has been delivered, the thread is severed.

51. A method according to claim 40, in which a stitching tool holds a plurality of anchors, an anchor delivery element advancing anchors one by one to successive penetrations.

52. A method according to claim 40, used in surgery.

53. A method according to claim 52, used for fixing soft tissue to soft tissue.

54. A method according to claim 53, in which separated soft tissue sections are approximated by pulling in the thread.

55. A method according to claim 52, used for fixing soft tissue to bone.

56. A method according to claim 55, in which at least one anchor is implanted in the bone.

57. A method according to claim 56, in which two anchors are implanted into bone through soft tissue to be fixed thereto and the suture thread extending between them tightened to hold the tissue against the bone.

58. A method according to claim 56, in which the bone is predrilled for the anchors.

59. An anchor and thread assembly comprising:
(i) a thread;
(ii) a first anchor threaded on the thread for securing a first end of the thread in a first penetration of a material to be stitched; and
(iii) a second anchor threaded on the thread for attaching the thread through a second penetration to the material;
wherein the first anchor and the second anchor are eyed;
wherein the second penetration is at a position spaced from the first penetration; and
wherein a second end of the thread is secured by a final anchor in a final penetration.

60. An assembly according to claim 59, of which the thread is a surgical suture thread and the first anchor is bio-compatible.

61. An assembly according to claim 59, provided in a cassette for attachment to a handpiece forming a stitching implement.

62. An assembly according to claim 59, wherein the first anchor has a hook-like configuration having a barb at at least one end and an eye for the thread.

63. An assembly according to claim 62, wherein the first anchor is made of bio-compatible material.

64. An assembly according to claim 63, wherein the first anchor is made of a bio-absorbable material.

65. An assembly according to claim 64, wherein the first anchor is made of an absorbable polymer such as polylactic acid or a co-polymer of PGA-co-TMC or of PGA-co-PCL.

66. An assembly according to claim 62, wherein the first anchor is made of metal.

67. An assembly according to claim 62, wherein the first anchor is made of a non-absorbable polymer such as acetal.

68. An assembly according to claim 62, wherein the first anchor comprises a releasable biological factor.

69. An assembly according to claim 68, wherein the first anchor includes a drug.

70. An assembly according to claim 68, wherein the first anchor includes an antibiotic.

71. An assembly according to claim 68, wherein the first anchor includes at least one of an inducer and a trigger for a natural healing factor.

72. A stitching implement comprising an anchor reservoir for a plurality of anchors and a thread reservoir for a thread and a piercing implement operative to penetrate material to be stitched and an anchor delivery element to deliver through a penetration one of said plurality of anchors with attached thread, the stitching implement being adapted to withdraw the piercing implement from the penetration, leaving the anchor, and to repeat the procedure at spaced apart locations along the material.

73. An implement according to claim 72, in which the anchor reservoir comprises a slideway on which anchors can be stacked.

74. An implement according to claim 72, in which the thread reservoir comprises a spool holder.

75. An implement according to claim 72, in which the anchor and thread reservoirs hold a plurality of anchor threads on a thread held in the thread reservoir.

76. An implement according to claim 72, comprising a thread cutting knife extendable from the region of the thread and anchor reservoirs.

77. An implement according to claim 72, fashioned as an endoscopic surgical device.

78. An implement according to claim 77, comprising an anchor indexing mechanism and a thread tension defining system operable from a handpiece.
